(19) United States
(12) Patent Application Publication
(10) Pub. No.: US 2012/0121730 A1
(43) Pub. Date: May 17, 2012

(54) PHARMACEUTICAL AND NUTRACEUTICAL COMPOSITIONS FOR TREATING RESPIRATORY DISEASE AND ASSOCIATED PHLEGM

(75) Inventor: Chandra U. Singh, San Antonio, TX (US)

(73) Assignee: Trinity Laboratories, Inc., San Antonio, TX (US)

(21) Appl. No.: 13/269,379

(22) Filed: Oct. 7, 2011

Related U.S. Application Data
(60) Provisional application No. 61/390,979, filed on Oct. 7, 2010.

(51) Int. Cl. A61K 31/7048; A61P 11/00; A61P 37/08; A61P 31/04; A61P 11/08; A61P 11/02; A61P 31/16; A61K 33/06; A61P 11/06

(52) U.S. Cl. 424/682; 514/27

(57) ABSTRACT

The present invention relates to pharmaceutical compositions comprising dextromethorphan or a physiologically acceptable salt thereof, quercetin, resveratrol, and hesperidin. The invention further relates to nutraceutical or dietary supplement composition comprising a physiologically acceptable salt of magnesium, quercetin, resveratrol, and hesperidin. Further provided are methods for treating respiratory disease in a human subject by substantially eliminating phlegm from the lung through administering to the subject effective amounts of compositions of the invention.

### CROSS REFERENCE TO RELATED APPLICATIONS

The application claims the benefit of U.S. Provisional Application No. 61/390,979, filed Oct. 7, 2010. The content of the aforesaid application is relied upon and is incorporated by reference herein in its entirety.

### BACKGROUND

1. Field of the Invention

The field relates to compositions for treating inflammatory afflictions, and in particular, inflammatory afflictions of the respiratory tract.

2. Description of Related Art

Excessive, thick phlegm or chest mucus is often caused by viral or bacterial infections such as influenza, bronchitis and pneumonia, as well as irritants such as those inhaled during smoking. Other possible causes also include allergies, Candida infection and Chronic Obstructive Pulmonary Disease.

Having excessive phlegm can be a very bothersome problem and may lead to hours of unrelenting, constant throat clearing and coughing. For some people, this is a temporary problem that lasts as long as the infection that causes it. For others however, excessive phlegm becomes an ongoing problem for which there is often little relief for how to best get rid of it. The alveolar and airway epithelium is recognized as a dynamic barrier that plays an important role in regulating the inflammatory and metabolic responses to oxidative stress, sepsis, endotoxemia, and other critical illnesses in the lung. The respiratory epithelium, in particular, is a primary target of an inflammatory/infectious condition at the epithelial-blood interface, and is itself capable of amplifying an inflammatory signal by recruiting inflammatory cells and producing inflammatory mediators.

Chronic Obstructive Pulmonary Disease (COPD) is one example of an inflammatory airway and alveolar disease where persistent upregulation of inflammation is thought to play a role. Inflammation in COPD is characterized by increased infiltration of neutrophils, CD8 positive lymphocytes, and macrophages into the airways. Neutrophils and macrophages play an important role in the pathogenesis of airway inflammation in COPD because of their ability to release a number of mediators including elastase, metalloproteases, and oxygen radicals that promote tissue inflammation and damage. It has been suggested that inflammatory cell accumulation in the airways of patients with COPD is driven by increased release of pro-inflammatory cytokines and of chemokines that attract the inflammatory cells into the airways, activate them d maintain their presence. The cells that are present also release enzymes (like metalloproteases) and oxygen radicals which have a negative effect on tissue and perpetuate the disease. A vast array of pro-inflammatory cytokines and chemokines have been shown to be increased within the lungs of patients with COPD. Among them, an important role is played by tumor necrosis factor alpha (TNF-alpha), granulocyte-macrophage colony stimulating factor (GM-CSF) and interleukin 8 (IL-8), which are increased in the airways of patients with COPD.

Other examples of respiratory diseases where inflammation seems to play a role include: asthma, eosinophilic cough, bronchitis, acute and chronic rejection of lung allograft, sarcoidosis, pulmonary fibrosis, rhinitis and sinusitis. Asthma is defined by airway inflammation, reversible obstruction and airway hyper responsiveness. In this disease, the inflammatory cells that are involved are predominantly eosinophils, T lymphocytes and mast cells, although neutrophils and macrophages may also be important. A vast array of cytokines and chemokines have been shown to be increased in the airways and play a role in the pathophysiology of this disease by promoting inflammation, obstruction and hyperresponsiveness.

Eosinophilic cough is characterized by chronic cough and the presence of inflammatory cells, mostly eosinophils, within the airways of patients in the absence of airway obstruction or hyperresponsiveness. Several cytokines and chemokines are increased in this disease, although they are mostly eosinophil directed. Eosinophils are recruited and activated within the airways and potentially release enzymes and oxygen radicals that play a role in the perpetuation of inflammation and cough.

Acute bronchitis is an acute disease that occurs during an infection or irritating event for example by pollution, dust, gas or chemicals, of the lower airways. Chronic bronchitis is defined by the presence of cough and phlegm production on most days for at least 3 months of the year, for 2 years. One can also find during acute or chronic bronchitis within the airways inflammatory cells, mostly neutrophils, with a broad array of chemokines and cytokines. These mediators are thought to play a role in the inflammation, symptoms and mucus production that occur during these diseases.

Sarcoidosis is a disease of unknown cause where chronic non-caseating granulomas occur within tissue. The lung is the organ most commonly affected. Lung bronchoalveolar lavage shows an increase in mostly lymphocytes, macrophages and sometimes neutrophils and eosinophils. These cells are also recruited and activated by cytokines and chemokines and are thought to be involved in the pathogenesis of the disease.

Pulmonary fibrosis is a disease of lung tissue characterized by progressive and chronic fibrosis (scarring) which will lead to chronic respiratory insufficiency. Different types and causes of pulmonary fibrosis exist but all are characterized by inflammatory cell influx and persistence, activation and proliferation of fibroblasts with collagen deposition in lung tissue. These events seem related to the release of cytokines and chemokines within lung tissue.

Acute rhinitis is an acute disease that occurs during an infection or irritating event, for example, by pollution, dust, gas or chemicals, of the nose or upper airways. Chronic rhinitis is defined by the presence of a constant chronic runny nose, nasal congestion, sneezing and pruritis. One can also find within the upper airways during acute or chronic rhinitis inflammatory cells with a broad array of chemokines and cytokines. These mediators are thought to play a role in the inflammation, symptoms and mucus production that occur during these diseases.

Acute sinusitis is an acute, usually infectious disease of the sinuses characterized by nasal congestion, runny, purulent phlegm, headache or sinus pain, with or without fever.

Chronic sinusitis is defined by the persistence for more than 6 months of the symptoms of acute sinusitis. One can also find during acute or chronic sinusitis within the upper airways and sinuses inflammatory cells with a broad array of chemokines and cytokines. These mediators are thought to play a role in the inflammation, symptoms and phlegm production that occur during these diseases.

As described above, these inflammatory respiratory diseases are all characterized by the presence of mediators that recruit and activate different inflammatory cells which release enzymes or oxygen radicals causing symptoms, the persistence of inflammation, and when chronic, destruction or disruption of normal tissue.

A logical therapeutic approach would be to downregulate cytokine and chemokine production and the inflammatory cell response. This has been performed in all the diseases described above by employing either topical or systemic corticosteroids with different levels of success. Corticosteroids are immune suppressive and have effects not only on inflammatory cells but also on other cells of the body that lead to toxicity when administered chronically.

Despite the availability of medications for COPD, asthma and other inflammatory respiratory diseases, the prevalence and morbidity of these diseases has remained stable or increased. There is an unmet medical need for the therapy of inflammatory respiratory diseases, and innovative therapeutic agents are urgently required. Antisense oligonucleotide-based therapy offers a new alternative approach to selectively decrease the expression of specific genes without the undesirable toxic effects of traditional therapeutic strategies. Antisense therapies are being investigated for the treatment of several diseases. It has been previously shown that antisense oligonucleotides directed against receptors for inflammatory mediators can be administered to the lungs and down-regulate their targets, as described in the published international patent application WO/1999/066037.

Administration of long-acting inhaled corticosteroids (ICS) in conjunction with long-acting beta-agonists (LABA) has been available for years for the treatment of asthma and COPD as a long term treatment. For example, the combination of budesonide (an ICS) and formoterol (a LABA) is available under the brand name SYMBICORT® and is recommended by the National Asthma Education and Prevention Program of the National

Institute of Health for long-term control and prevention of symptoms of moderate and severe persistent asthma. The combination is offered in a dry powder inhaler device marketed as the TURBUHALER® by AstraZeneca.

The list of potential side effects of corticosteroids is long and includes the following: increased appetite and weight gain; deposits of fat in chest, face, upper back, and stomach; water and salt retention leading to swelling and edema; high blood pressure; diabetes; black and blue marks; slowed healing of wounds; osteoporosis; cataracts; muscle weakness; thinning of the skin; increased susceptibility to infection; stomach ulcers; increased sweating; mood swings; psychological problems such as depression; and adrenal suppression and crisis.

Allergy shots, also called “immunotherapy,” are given to increase tolerance to the substances (allergens) that provoke allergy symptoms. They usually are recommended for patients who suffer from severe allergies or for those who have allergy symptoms more than three months each year. They do not cure allergies, but reduce a patient's sensitivity to certain substances.

Leukotriene modifiers block the effects of leukotrienes, immune system chemicals that cause asthma symptoms. Leukotriene modifiers can help prevent symptoms for up to 24 hours. Examples of such modifiers include montelukast (SINGULAIR®); zafirlukast (ACCOLATE®); and zileuton (ZYFLO®, ZYFLO CR®).

A therapeutic approach that would decrease pro-inflammatory cytokine and chemokine release by a vast array of cells while having a reduced effect on the release of anti-inflammatory mediators or enzymes may have an advantage over current therapies for inflammatory respiratory diseases or any other systemic inflammatory disease.

In the publication, “Dietary Fiber and Reduced Cough with Phlegm,” American Journal of Respiratory and Critical Care Medicine Vol. 170. pp. 279-287, (2004), L. M. Butler et al. summarize issues with cough and phlegm as follows:

“Cough and phlegm are frequently associated with chronic obstructive pulmonary disease, which may be caused by oxidative stress-mediated inflammation and tissue damage in the lung. Oxidants can cause direct damage by inactivating antiproteases or mediating other processes that promote the development of chronic lung damage . . . . There is evidence that dietary nutrients modulate oxidative stress-induced lung damage among both smokers and nonsmokers. Fruits and vegetables are the major food sources of antioxidants that may protect the lung from oxidative stress. Fruit intake and, to a lesser extent, vegetable intake have been associated with higher lung function and reduced symptoms of cough with phlegm. Cross-sectional studies support an association between vitamin C and lung function. However, the epidemiologic evidence is stronger for fruit intake than for individual fruit-related nutrients such as vitamin C and carotenoids, suggesting that other nutrients associated with fruit may be more relevant in protecting the lung from oxidative stressors. In addition to vitamin C and carotenoids, fruit and vegetables contain flavonoids and fiber. Flavonoids have free-radical scavenging properties that may influence lung disease, but have rarely been evaluated in prospective studies. Some epidemiologic evidence suggests that flavonoid-containing fruits, such as apples and pears, are associated with improved lung function and reduced nonspecific respiratory disease. Reduced chronic obstructive pulmonary disease symptoms were also reported among individuals with greater intake of the flavonoids known as catechins. In addition to fruit and vegetables, flavonoids are found in soyfoods, tea, and wine.”

The most important polyphenol compounds are the flavonoids, which are abundant components of the human diet. Quercetin, a key representative flavonoid molecule of the group, is present widely in vegetables and fruits, with a daily intake of up to 25 mg/day in a normal human diet. Other effects such as antitumoral, antithrombotic, anti-inflammatory and antiapoptotic ones, as well as effects inhibiting platelet aggregation and the growth of certain types of cancer, have been described for quercetin and other flavonoids.

As disclosed by Y. Yamamoto et al. in J. Clin. Invest. 2001;107(2):135-142, “Flavonoids . . . exhibit a variety of biological activities, including suppression of inflammation, cancer chemoprevention, and protection from vascular disease. Several reports suggest that the properties of the flavonoids quercetin, resveratrol, and myricetin may be mediated through downregulation of the NF-κB pathway. For example, resveratrol, which is found in red wine, can inhibit NF-κB activity and induce apoptosis in transformed cells, which may contribute to the ability of red wine to reduce mortality from coronary heart diseases and certain cancers. Resveratrol has strong inhibitory effects on iNOS expression and NO generation in activated macrophages. Treatment of macrophages with this compound blocks LPS-induced phosphorylation and degradation of IκBα to decrease NF-κB DNA binding activity, suggesting that its anti-inflammatory effects may be due at least in part to the inhibition of NF-κB-dependent NO synthesis. The inhibitory effects of resveratrol and the flavonoid myricetin on activation of the NF-κB pathway correlate with their ability to reduce IKK activity. Thus several of the biological activities of flavonoids may be mediated by their inhibition of the NF-κB pathway.”

Glutamate is an excitatory amino acid. With regard to glutamate, C. J. Ma et al. disclose in Br. J. Pharmacol. 2005 November; 146(5): 752-759, “Glutamate . . . activates different types of ion channel-forming receptors and G-protein-coupled receptors and plays its essential roles—neuronal survival, synaptogenesis, neuronal plasticity, memory, learning and behavior—in the central nervous system (CNS). However, high concentration of glutamate cause neuronal cell death within the CNS, and may be involved in neuropsychiatric and neuropathological disorders such as Parkinson's disease, Alzheimer's disease, epilepsy, seizures, ischemic stroke and spinal cord trauma. Thus, neuroprotection against glutamate-induced neurotoxicity has been a therapeutic strategy to treat neurodegenerative diseases.”

“All living cells have developed mechanisms for protection against oxidative stress. In general, GSH plays a major role in the elimination of a large number of nucleophilic toxicants such as oxidative radicals. In normal cells, GSH levels are decreased by oxidative radicals but are promptly restored to normal levels. The depletion of GSH alone did not result in a severe leakage of LDH from primary cultured cells; however, glutamate insult to cells rapidly and continuously decreased cellular GSH levels and inactivated many related antioxidant enzymes including superoxide dismutase, GSH-px and GSH-R. Thus, toxic free radicals such as O₂⁻,H₂O₂ were kept high in response. Such defects in GSH metabolism ght cause oxidative stress, which has been implicated in several neurologic and neurodegenerative diseases.” MDGA (Meso-dihydroguaiaretic acid) and Licarin A diminished the calcium influx that routinely accompanies with the glutamate-induced neurotoxicity, and inhibited the subsequent overproduction of cellular nitric oxide and peroxide to the level of control cells. They also preserved cellular activities of antioxidative enzymes such as superoxide dismutase, glutathione peroxidase and glutathione reductase reduced in the glutamate-injured neuronal cells.

Dextromethorphan (frequently abbreviated as DM) is the generic name for (+)-3-methoxy-N-methylmorphinan, which is shown in FIG. 1. As disclosed in U.S. Pat. No. 5,350,756 to Smith, it widely used as a cough syrup, and is described in references such as in Chemistry of Carbon Compounds by E. H. Rodd, Elsevier Publishing 1960, and Goodman & Gilman's The Pharmacological Basis of Therapeutics, Chapter 21, “Opiate Analgesics and Antagonists”, pp. 485-521 (8^{th} ed. 1990), which is incorporated herein by reference.

DM is a non-addictive opioid comprising a dextrorotatory enantiomer (mirror image) of the morphinan ring structure which forms the molecular core of most opiates. DM acts at a class of neuronal receptors known as sigma receptors. These are often referred to as sigma opiate receptors, but there is some question as to whether they are opiate receptors, so many researchers refer to them simply as sigma receptors, or as high-affinity dextromethorphan receptors. They are inhibitory receptors, which mean that their activation by DM or other sigma agonists causes the suppression of certain types of nerve signals. Dextromethorphan also acts at another class of receptors known as N-methyl-D-aspartate (NMDA) receptors, which are one type of excitatory amino acid (EAA) receptor. Unlike its agonist activity at sigma receptors, DM acts as an antagonist at NMDA receptors, which means that DM suppresses the transmission of nerve impulses mediated via NMDA receptors. Since NMDA receptors are excitatory receptors, the activity of DM as an NMDA antagonist also leads to the suppression of certain types of nerve signals, which may also be involved in some types of coughing.

Due to its activity as an NMDA antagonist, DM and one of its metabolites, dextrorphan, are being actively evaluated as possible treatments for certain types of excitotoxic brain damage caused by ischemia (low blood flow) and hypoxia (inadequate oxygen supply), which are caused by events such as stroke, cardiac arrest, and asphyxia. The anti-excitotoxic activity of dextromethorphan and dextrorphan, and the blockade of NMDA receptors by these drugs, are discussed in items such as Choi (Choi D W. Dextrorphan and dextromethorphan attenuate glutamate neurotoxicity. Brain Res 1987; 403: 333-6), Wong et al (Wong B Y, Coulter D A, Choi D W, Prince D A. Dextrorphan and dextromethorphan, common antitussives, are antiepileptic and antagonize N-methyl-D-aspartate in brain slices. Neurosci Lett. 1988 Feb. 29; 85(2):261-266) and Steinberg et al (Steinberg G K et al, Delayed treatment with dextromethorphan and dextrorphan reduces cerebral damage after transient focal ischemia, Neurosci Letters 1988; 89: 193-197) and U.S. Pat. No. 4,806,543. Dextromethorphan has also been reported to suppress activity at neuronal calcium channels (Carpenter C L et al., Dextromethorphan and dextrorphan as calcium channel antagonists, Brain Research 1988; 439: 372-375). Dextromethorphan and the receptors it interacts with are further discussed in Tortella et al. (Tortella F C, Pellicano M, Bowery N G. Dextromethorphan and neuromodulation: old drug coughs up new activities. Trends Pharmacol Sci. 1989 December; 10(12):501-507), Leander et al. (Leander J D, Rathbun R C, Zimmerman D M. Anticonvulsant effects of phencyclidine-like drugs: relation to N-methyl-D-aspartic acid antagonism. Brain Res. 1988 Jun 28;454(1-2):368-372), Koyuncuoglu & Saydam (Koyuncuoglu and Saydam. Intnl J Clin Pharmacol Ther Tox 1990; 28: 147-152), Ferkany et al. (Ferkany J W, Borosky S A, Clissold D B, Pontecorvo M J, Dextromethorphan inhibits NMDA-induced convulsions. Eur J Pharmacol. 1988 Jun. 22; 151(1):151-154), Prince & Feeser (Prince & Feeser. Neurosci Letters 1988; 85: 291-296) and Musacchio et al. (Musacchio J M et al., High affinity dextromethorphan binding sites in the guinea pig brain, J Pharmacol Exp Ther 1988; 247: 424-431).

DM disappears fairly rapidly from the bloodstream (see, e.g., Vettican S J et al., Phenotypic differences in dextromethorphan metabolism, Pharmaceut Res 1989; 6: 13-19). DM is converted in the liver to two metabolites called dextrorphan and 3-methoxymorphinan, by an enzymatic process called O-demethylation; in this process, one of the two pendant methyl groups is replaced by hydrogen. If the second methyl group is removed, the resulting metabolite is called 5-hydroxymorphinan. Dextrorphan and 5-hydroxymorphinan are covalently bonded to other compounds in the liver (primarily glucuronic acid or sulfur-containing compounds such as glutathione) to form glucuronide or sulfate conjugates which are eliminated fairly quickly from the body via urine bloodstream.

In summary, Dextromethorphan and its active metabolite dextrorphan bind to the N-Methyl-D-Aspartate (NMDA) glutamate and nicotine/neuronal nicotinic receptors as inhibitors. Dextromethorphan and dextrorphan also bind to the receptor-gated (NMDA receptor mediated) and voltage-gated calcium channels, and the voltage-gated sodium channels as a blocker. Through these bindings, dextromethorphan and dextrorphan modulates the glutamate pathway in the central nervous system (CNS) and modulate most of the excitatory synaptic transmission. Dextromethorphan and dextrorphan also bind to the sigma receptors which are found in high concentrations in limbic and motor areas of the CNS sensory processing such as the dorsal root ganglia and the nucleus tractus solitarus (NTS). In addition, Dextromethorphan inhibits the reuptake of 5-HT (serotonin) and norepinephrine, thus modulating the monamine pathways. Magnesium participates in numerous enzymatic reactions including all reactions that involve the formation and utilization of adenosine-50-triphosphate (ATP) in energy metabolism (http://lpi.oregonstate.edu/infocenter/minerals/magnesium/). Whenever neurons cannot generate sufficient ATP to keep their ion pumps working properly, membranes depolarize and excessive Ca2+ leaks into cells, triggering the synaptic release of glutamate, which further depolarizes neurons, further raising intracellular Ca2+ which causes even more glutamate to be released repeating in endless cycles resulting in neuronal dysfunction and depression.

Magnesium has been shown to cause a dose-dependent inhibition of platelet aggregation. Magnesium has a strong vascular dilating effect, lending support to the vascular theory of migraine. IMg2+ levels are known to affect entry of Ca2+, and intracellular ICa2+ from sarcoplasmic and endoplasmic reticulum, in vascular smooth muscle and vascular endothelial cells and to control vascular tone and reactivity to endogenous hormones and neurotransmitters. Cerebral blood vessel muscle cells are particularly sensitive to IMg2+; Mg deficiency results in contraction and potentiation of vasoconstrictors and excess IMg2+ results in vasodilatation and inhibition of vasoconstrictors.

Magnesium is intimately involved in the control of N-methyl-D-'aspartate (NMDA) glutamate receptors which play an important role in pain transmission in the nervous system and regulation of cerebral blood flow (Iseri L T, French J H. Magnesium: nature's physiologic calcium blocker. Am Heart J 1984; 108:188-93). Magnesium ion plugs the NMDA receptor and prevents calcium ions from entering the cell. Lowering Mg2+ concentration facilitates activation of the NMDA receptor, which allows calcium to enter the cell and exert its effects both on neurons and cerebral vascular muscle. Thus magnesium can be considered an NMDA receptor antagonist at several important sites.

Magnesium is involved in many central nervous processes both at presynaptic and postsynaptic levels. Changes in magnesium concentration exert diverse influences on neurons, in normal or pathological conditions (Yasui M, Ota K, Murphy V A. Magnesiu related neurological disorders. In: Yasui M, Strong M J, Ota K, Verity M A, editors. Mineral and metal neurotoxicology. Boca Raton, Fla.: CRC Press LLC; 1997. p. 219-26 [chapter 22]). Resveratrol, also known as trans-3,4′,5-trihydroxystilbene, and shown in FIG. 2,is a naturally occurring plant antibiotic known as phytoalexins and is found in many plants, nuts, and fruits and is abundant in grapes and red wine. Its function is to protect the plant from injury, UV irradiation, and fungal attack. Many studies have demonstrated that resveratrol has a wide range of pharmacological properties, which have been comprehensively reviewed by Bhat et al. (Bhat, K. P. L., Kosmeder, J. W., and Pezzuto, J. M. (2001) Biological effects of resveratrol. Antioxid. Redox Signal. 3, 1041-1064). Resveratrol has been suggested to be cardio-protective via various mechanisms such as its antioxidant activity, inhibition of platelet aggregation, induction of NO production, and modulation of the synthesis of hepatic apolipoprotein and lipids. Resveratrol is also reported to have chemo-preventive activity; Jang et al. (Jang, M., Cai, L., Udeani, G. O., Slowing, K. V., Thomas, C. F., Breecher, C. W., Fong, H. H., Farnsworth, N. R., Kinghorn, A. D., Mehta, R. G., et al. (1997) Cancer chemopreventive activity of resveratrol, a natural product derived from grapes. Science 275, 218-220) have suggested that it inhibits all three phases of tumor development: initiation, promotion, and progression. Considerable evidence demonstrates anti-inflammatory properties of resveratrol, including inhibition of reactive oxygen species in neutrophils (Rotondo, S., Rajtar, G., Manarini, S., Celardo, A., Rotillo, D., De Gaetano, G., Evangelista, V., and Cerletti, C. (1998) Effect of trans-resveratrol, a natural polyphenolic compound, on human polymorphonuclear leukocyte function. Br. J. Pharmacol. 123, 1691-1699), monocytes, and macrophages. Rotondo et al. showed that resveratrol reduced elastase and β-glucuronidase secretion and β2 integrin MAC-1 expression on neutrophils. Other groups have published on the inhibitory effect of resveratrol on the expression of the adhesion molecules ICAM-1, VCAM-1 (Intercellular adhesion molecule-1, vascular cell adhesion molecule-1), and E-selectin on endothelial cells. Ferrero et al. (Ferrero, M. E., Bertelli, A. A. E., Fulgenzi, A., Pellegatta, F., Corsi, M. M., Bonfrate, M., Ferrara, F., De Caterina, R., Biovannini, L., and Bertelli, A. (1998) Activity in vitro of resveratrol on granulocyte and monocyte adhesion to endothelium. Am. J. Clin. Nutr. 68, 1208-1214) also reported that resveratrol reduced granulocyte and monocyte adhesion to endothelial cells. The release of various cytokines from macrophages and lymphocytes, such as IL-6, IFNγ, IL-2, TNF-α, and IL-12, has been shown to be inhibited by resveratrol (Gao, X., Xu, Y. X., Janakiraman, N., Chapman, R. A., and Gautam, S. C. (2001) Immunomodulatory activity of resveratrol: suppression of lymphocyte proliferation, development of cell-mediated cytotoxicity, and cytokine production. Biochem. Pharmacol. 62, 1299-1308). In stimulated macrophages the expression of iNOS and the release of nitric oxide are reduced by resveratrol. A reduction of cyclooxygenase (COX)-1 and COX-2 expression and activity by resveratrol is also apparent (Cho, D., Koo, N.-Y., Chung, W. J., Kim, T.-S., Ryu, S. Y., Im, S. Y., and Kim, K. M. (2002) Effects of resveratrol-related hydroxystilbenes on the nitric oxide production in macrophages cells: structural requirements and mechanism of action. Life Sci. 71, 2071-2082). A common link between the inhibitory effects of resveratrol mentioned above could be its ability to inhibit factors involved in gene transcription like MAPK, c-JNK, AP-1, and NF-κB. Many of the inflammatory biomarkers reported to be impacted on by resveratrol in vitro are known to be implicated in LPS-induced airway inflammation, including NO; TNF-α; IL-1β; the IL-8/GROα equivalent in rats CINC 1, 2, and 3; P and L selectin; IL-6; and VCAM-1 (McCluskie, K., Birrell, M. A., Wong S. and Belvisi, M. G. (2004) Nitric oxide as a non-invasive biomarker of LPS induced airway inflammation: Possible role in lung neutrophilia. J. Pharmacol. Exp. Ther, 311, 625-633). This study aimed to determine the effect of resveratrol, in parallel with a glucocorticoid-positive control, on LPS-induced airway inflammation in the rat. Resveratrol has low oral bioavailability; hence it has been suggested that regular consumption of resveratrol, i.e., through frequently drinking moderate amounts of red wine, is necessary to achieve the beneficial effects on health. It is not known what length of time is necessary to orally consume resveratrol to achieve the possible anti inflammatory affects. Hesperidin, also known as HES, or 5,7,3′-trihydroxy-4′-methoxy-flavanone-7-rhamnoglucoside, assigned CAS number 520-26-3, and shown in FIG. 3, is a flavanone-type flavonoid. Hespiridin is abundant in citrus fruit and has been reported to possess significant anti-inflammatory, analgesic, antioxidant, antifungal and antiviral activities. A study reports that HES could reduce TNF-[alpha] production and inhibits infection-induced lethal shock (Kawaguchi, K., Kikuchi, S., Masunuma, R., Maruyama, H., Yoshikawa, T., Kumazawa, Y., 2004. A citrus flavonoid hesperidin suppresses infectioninduced endotoxin shock in mice. Biol. Pharm. Bull. 27, 679-683). The mechanisms for those effects are not clear, but the results of the aforementioned studies support the concept that HES could be an immunomodulator agent in severe systemic inflammation. Although the anti-inflammatory effects of HES in rat colitis induced by TNBS has been reported (Crespo, M. E., Galvez, J., Cruz, T., Ocete, M. A., Zarzuelo, A., 1999. Anti-inflammatory activity of diosmin and hesperidin in rat colitis induced by TNBS. Planta Med. 65, 651-653; Lei Xu, Zhone-lin Yang, Ping Li, Yong-qiang Zhou. Modulating effect of Hesperidin on experimental murine colitis induced by dextran sulfate sodium. doi:10.1016/j.phymed.2009.02.021), there is no information regarding the influence of HES treatment on ulcerative colitis as an immuno-modulator.

Hesperidin has antioxidant, anti-inflammatory, hypolipidemic, vasoprotective and anticarcinogenic and cholesterol lowering actions. Hesperedin can inhibit the following enzymes: phospholipase A2, lipoxygenase, HMG-CoA reductase and cyclo-oxygenase. Hesperidin improves the health of capillaries by reducing the capillary permeability. Hesperidin is used to reduce hay fever and other allergic conditions by inhibiting the release of histamine from mast cells. The possible anti-cancer activity of hesperidin could be explained by the inhibition of polyamine synthesis.

Quercetin, also known as 3,3′,4′,5,7-pentahydroxyflavone, and shown in FIG. 4, is a dietary flavonoid found in many plants including onions, broccoli, apples, berries, and tea. Quercetin is the major flavonoid in the human diet, with an estimated average dietary intake in the United States of 25 mg/d. It is also present in extracts from Gingko biloba and St. John's Wort, both popular health supplements.

Quercetin actively participates in intracellular signaling, inhibiting phosphatidylinositol-3 kinase, protein kinase C, xanthine oxidase and NADPH diaphorase. Nevertheless, in spite of this multiplicity of actions, the cardiovascular and/or neuroprotective effects of flavonoids and quercetin are mainly explained by their antioxidant capacity and their ability to scavenge free radicals.

Quercetin has been found to have inhibitory effects on several lipid, protein tyrosine, and serine/threonine kinases, including phosphatidylinositol (PI) 3-kinase, AMP-activated kinase, casein kinase 2, p90 ribosomal protein S6 kinase, p70 ribosomal S6 kinase, protein kinase C, epidermal growth factor receptor tyrosine kinase, and IκB kinase.

As disclosed by S. Nanua in American Journal of Respiratory Cell and Molecular Biology. Vol. 35, pp. 602-610, 2006, quercetin has been shown to have potent effects in diverse biological systems. These include antiproliferative and proapoptotic effects for many cancer or preneoplastic cell lines, anti-inflammatory effects, and protection against oxidative stress. Focusing on the potential benefit of quercetin in the treatment of airways disease, several studies have demonstrated an inhibitory effect on cytokine or chemokine production in cultured cells. In one system, quercetin has been shown to attenuate lipopolysaccharide-induced nitric oxide production, inducible nitric oxide synthase expression, and release of TNF-α and IL-6 in RAW 264.7 macrophages. In these studies, quercetin strongly reduced activation of mitogen-activated protein kinases and NF-κB, a transcription factor complex known to play a critical role in the expression of proin-flammatory genes. Further studies have showed that luteolin, a related flavonoid, interfered with the phosphorylation of Akt, a downstream effector of PI 3-kinase, as well as NF-κB activation. Quercetin has also been noted to have inhibitory effects on mast cell activation and release of histamine, TNF-α, IL-6, and IL-8 (Kempuraj D, Madhappan B, Christodoulou S, Boucher W, Cao J, Papadopoulou N, Cetrulo C L, Theoharides T C. Flavonols inhibit proinflammatory mediator release, intracellular calcium ion levels and protein kinase C theta phosphorylation in human mast cells. Br J Pharmacol 2005; 145:934-944). Quercetin inhibits the induction of IL-8 and monocyte chemoattractant protein (MCP)-1 by TNF-α in cultured human synovial cells (Sato M, Miyazaki T, Kambe F, Maeda K, Seo H. Quercetin, a bioflavonoid, inhibits the induction of interleukin 8 andmonocyte chemoattractant protein-1 expression by tumor necrosis factor-alpha in cultured human synovial cells. J Rheumatol 1997; 24:1680-1684). EM-X, a quercetin-containing mixture derived from the ferment of unpolished rice, papaya, and seaweed, inhibits both H2O2- and TNF-α-induced IL-8 expression in cultured human alveolar epithelial A549 cells (Deiana M, Assunta Dessi M, Ke B, Liang Y-F, Higa T, Gilmour P S, Jen L-S, Rahman I, Anima O I. The antioxidant cocktail effective microorganism X (EM-X) inhibits oxidant-induced interleukin-8 release and the peroxidation of phospholipids in vitro. Biochem Biophys Res Commun 2002; 296:1148-1151). Since TNF-α, a proinflammatory cytokine; IL-8, a potent C—X—C chemokine with the neutrophil chemoattracting E-L-R motif; and MCP-1, a C—C (α) chemokine shown to be an important mediator of monocyte and CD4+/CD8+ lymphocyte recruitment, are each increased in the airways of patients with asthma, these data are consistent with the notion that quercetin may reduce airway inflammation in this disease.

Heretofore, a number of patents and publications have disclosed compositions and treatment regimens to address inflammatory diseases. These diseases may be of the respiratory tract including, for example, asthma, chronic obstructive pulmonary disease (COPD), acute respiratory distress syndrome, bronchitis, chronic bronchitis, silicosis, pulmonary fibrosis, lung allograft rejection, allergic rhinitis and chronic sinusitis. These diseases may be skin conditions, disorders or diseases, such as may be associated with or caused by inflam nation, sun damage or natural aging, or they may be associated with a dietary phytochemical deficiency. Such patents and published applications include U.S. Pat. Nos. 7,579,455, 6,414,037, 6,979,689, 6,270,780, 7,745,487, and 6,878,751; and U.S. Patent Application Publication Nos. 20020025350, and 20090087425, the disclosures of which are incorporated herein by reference.

In spite of the compositions and treatment regimens disclosed in these patent and published applications, and in spite of the numerous related pharmaceutical products currently on the market, there remains a need for a pharmaceutical composition which can significantly reduce or eliminate inflammatory afflictions, such as allergy symptoms and phlegm in the lung of patients who are suffering from asthma and severe allergy.

### SUMMARY

The present invention meets this need by providing various pharmaceutical compositions and methods which address these afflictions.

In one aspect recited in the present disclosure, a pharmaceutical composition is provided comprising dextromethorphan or a physiologically acceptable salt thereof, quercetin, resveratrol, and hesperidin. In some embodiments, the weight ratio between dextromethorphan or the physiologically acceptable salt thereof, quercetin, resveratrol, and hesperidin is 1:0.2-5:0.2-5.0: 0.2-5.0.

In another aspect, a pharmaceutical composition is provided comprising dextromethorphan or a physiologically acceptable salt thereof, quercetin, resveratrol, and hesperidin, in which a weight ratio dextromethorphan or the physiologically acceptable salt thereof, quercetin, resveratrol, and hesperidin is 1:0.2-5:0.2-5.0:0.2-5.0.

In yet another aspect, a pharmaceutical composition is provided consisting essentially of dextromethorphan or a physiologically acceptable salt thereof, quercetin, and resveratrol. The term “consisting essentially of,” when used herein is meant to express a composition as including the recited ingredients and those that do not materially affect the basic and novel characteristics of the composition, such as the efficacy of the composition in treating one or more target conditions described herein (e.g., respiratory disease, elimination of phlegm from the lung, cough and allergy). An example of such a pharmaceutical composition contains the just-mentioned three ingredients and a pharmaceutically acceptable carrier. Another example is a soft chew composition containing the four ingredients and various inactive additives (e.g., excipients, sweeteners, and artificial flavors).

The pharmaceutical composition may be either in dry form (e.g., powder or tablet) or in liquid form (e.g., syrup). In some embodiments, the pharmaceutical formulation may be in the form of a tablet, a capsule, a soft chew, or a gel.

Yet in another aspect recited in the present disclosure, a composition is provided comprising a physiologically acceptable salt of magnesium, quercetin, resveratrol, and hesperidin. In some embodiments, the weight ratio between the physiologically acceptable salt of magnesium, quercetin, resveratrol, and hesperidin is 1:0.2-1:0.2-1.0:0.2-1.0.

Yet in another aspect, a composition is provided consisting essentially of a physiologically acceptable salt of magnesium, quercetin, resveratrol, and hesperidin. An example of such a composition contains the just-mentioned four ingredients and a pharmaceutically acceptable carrier. Another example is a soft chew composition containing the four ingredients, optionally the carrier, and various inactive additives (e.g., excipients, sweeteners, and artificial flavors).

The composition, either in dry form (e.g., powder or tablet) or in liquid form (e.g., beverage or syrup), can be a dietary supplement or a pharmaceutical formulation. In some embodiments, the dietary supplement or the pharmaceutical formulation can be in the form of a tablet, a capsule, a soft chew, or a gel. The composition can also be a food product. Examples include tea (e.g., a tea drink and the contents of a tea bag), soft drinks, juice (e.g., a fruit extract and a juice drink), milk, coffee, jelly, ice cream, yogurt, cookies, cereals, chocolates, and snack bars.

Yet in another aspect, a method for treating respiratory disease is provided for substantially eliminating phlegm from the lung, allergy symptoms and cough. In some embodiments, the method comprises administering to a subject in need thereof an effective amount of the above-described compositions. Examples of removing the phlegm from the lung include expelling the phlegm already present in the congested chest and protecting the lung from producing more mucus. Examples of respiratory lung disease include chest congestion produced by viral or bacterial infections such as influenza, and pneumonia, allergies, asthma, COPD, eosinophilic cough, bronchitis, acute and chronic rejection of lung allograft, sarcoidosis, pulmonary fibrosis, rhinitis and sinusitis. By properly administrating any of the compositions, a subject's respiratory condition can be greatly enhanced without deleterious side effects.

In yet another aspect, a method for treating a disorder associated with oxidative stress (e.g., respiratory lung disease, diabetic neuropathy, neurodegenerative disease) is provided comprising administering to a subject in need thereof an effective amount of a composition of the invention.

In a further aspect, a method of administering to a subject in need thereof an effective amount of the above-described composition is provided to treat one or more of the following diseases: autoimmune disease, inflammatory disease, arthritis, tumorₛ diabetes, chronic constipation, cold, viral and bacterial infection (e.g., upper respiratory tract infection), or neurodegenerative disease (e.g., age-related brain degenerative disease).

Also within the scope of this invention is a composition containing the composition described above for use in treating the above-described disorders or conditions, and the use of such a composition for the manufacture of a medicament for the just-mentioned treatment.

The details of one or more embodiments of the invention are set forth in the description below. Other features, objects, and advantages of the invention will be apparent from the description and from the claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 depicts the chemical structure of dextromethorphan.

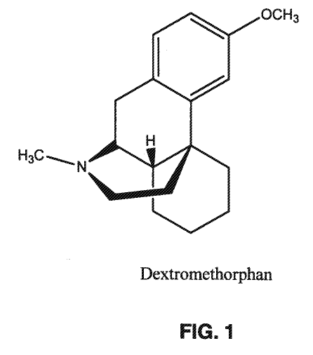

FIG. 2 depicts the chemical structure of resveratrol.

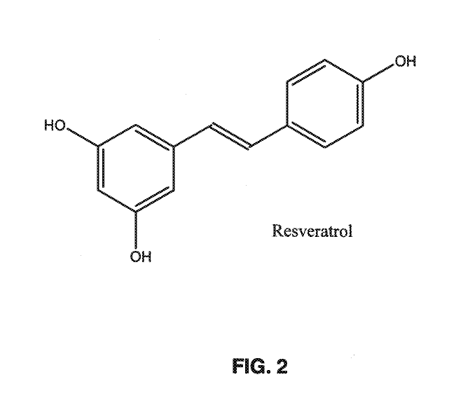

FIG. 3 depicts the chemical structure of hesperidin.

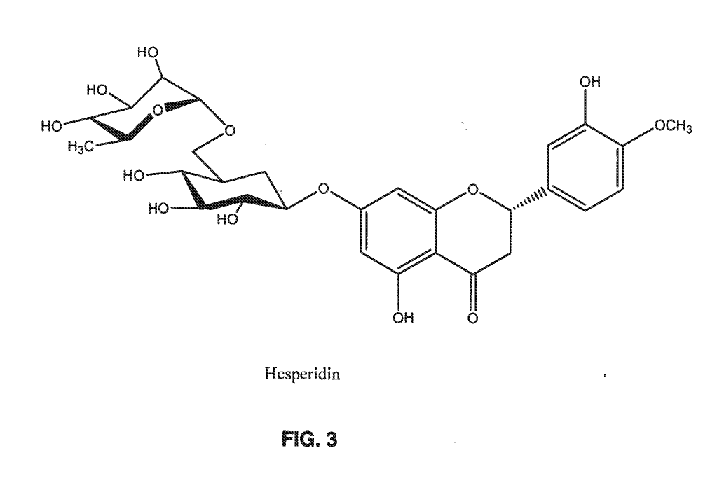

FIG. 4 depicts the chemical structure of quercetin.

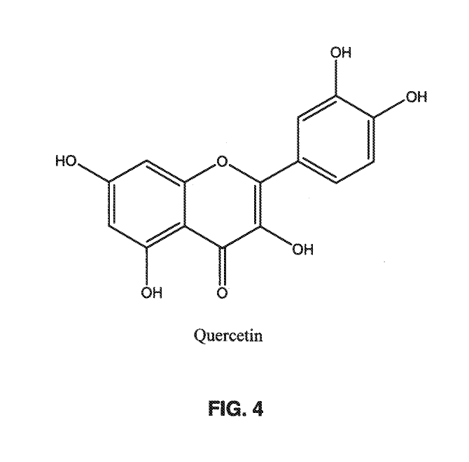

FIG. 5 depicts the chemical structure of Licarin-A.

FIG. 6 depicts the chemical structure of Naringin.

FIG. 7 depicts the chemical structure of Myricetin.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DETAILED DESCRIPTION

Before the present invention is described in detail, it is to be understood that unless otherwise indicated this invention is not limited to any particular formulation, carrier, or drug administration regimen, as such may vary. It is also to be understood that the terminology used herein is for the purpose of describing particular embodiments only and is not intended to limit the scope of the present invention.

In this specification and in the claims which follow, reference will be made to a number of terms which shall be defined to have the following meanings:

must be noted that as used herein and in the claims, the singular forms “a,” “and” and “the” include plural referents unless the context clearly dictates otherwise. Thus, for example, reference to “an active agent or ingredient” in a formulation includes one, two, or more active agents or ingredients, reference to “a carrier” includes one, two, or more carriers, and so forth.

The terms “active agent,” “active ingredient,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By “pharmaceutically acceptable carrier” is meant a material or materials that are suitable for drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable or physiologically acceptable” salt, ester, or other derivative of an active agent as provided herein is a salt, ester, or other derivative that is not biologically or otherwise undesirable.

By “pharmaceutical composition” is meant a formulation containing at least one ingredient such as dextromethorphan which can not be classified as a nutraceutical or food.

By “composition” is meant a formulation which can be classified either as a pharmaceutical formulation or a nutraceutical formulation depending upon its use.

By the terms “respiratory disease”, or “respiratory lung disease” or “inflammatory lung disorder” or “respiratory disorder” or “inflammatory lung disorder” as provided herein are meant to describe chest congestion and mucus formation in the pulmonary airways produced by viral or bacterial infections, such as influenza or pneumonia; or produced by allergies, asthma, COPD, eosinophilic cough, bronchitis, acute and chronic rejection of lung allograft, sarcoidosis, pulmonary fibrosis, rhinitis or sinusitis.

By the terms “effective amount” or “therapeutically effective amount” of a composition as provided herein are meant a nontoxic but sufficient amount of the composition to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” asthma, as the term “treating” is used herein, encompasses both prevention of asthma in a predisposed individual and treatment of asthma in a clinically symptomatic individual.

The terms “condition,” “disease,” and “disorder” are used interchangeably herein as referring to a physiological state that can be prevented or treated by administration of a pharmaceutical formulation as described herein.

The term “patient” as in treatment of “a patient” refers to a mammalian individual afflicted with or prone to a condition, disease, or disorder as specified herein, and includes both humans and animals.

The term “pulmonary” as used herein refers to any part, tissue or organ that is directly or indirectly involved with gas exchange, i.e., O₂/CO₂ exchange, within a patient. “Pulmonary” contemplates both the upper and lower airway passages and includes, for example, the mouth, nose, pharynx, oropharynx, laryngopharynx, larynx, trachea, carina, bronchi, bronchioles and alveoli.

The term “resveratrol” is intended to mean either the cis-isomer of resveratrol, the trans-isomer of resveratrol, or a mixture of the two isomers. The term is also intended to include both the naturally occurring active agent and the compound as it may be chemically synthesized in the laboratory. Further, when the term “resveratrol” is used herein, it is intended to encompass pharmacologically acceptable salts, esters, amides, prodrugs and analogs of resveratrol.

The term “quercetin” is intended to include both the naturally occurring active agent and the compound as it may be chemically synthesized in the laboratory. Further, when the term “quercetin” is used herein, it is intended to encompass pharmacologically acceptable salts, esters, amides, prodnigs, analogs of quercetin and glycoside quercetin derivatives, e.g., quercetin-3-O-glucoside, quercetin-5-O-glucoside, quercetin-7-O-glucoside, quercetin-9-O-glucoside, quercetin-3-O-rutinoside, quercetin-3-O[α-rhamnosyl-(1→2)-α-rhamnosyl-(1→6)]-β-glucoside, quercetin-3-O-galactoside, quercetin-7-O-galactoside, quercetin-3-O-rhamnoside, and quercetin-7-O-galactoside.

The term “hesperidin” is intended to include both the naturally occurring active agent, its active metabolite hesperetin, and the compound as it may be chemically synthesized in the laboratory. Further, when the term “hesperidin” is used herein, it is intended to encompass pharmacologically acceptable salts, esters, amides, prodrugs and analogs of hesperidin.

“Optional” or “optionally” means that the subsequently described circumstance may or may not occur, so the description includes instances where the circumstance occurs and instances where it does not. For example, recitation of an additive as “optionally present” in a formulation herein encompasses both the formulation containing the additive and the formulation not containing the additive.

The term “food” broadly refers to any kinds of liquid and solid/semi-solid materials that are used for nourishing humans and animals, for sustaining normal or accelerated growth, or for maintaining stamina or alertness.

The term “C-reactive protein” refers to a protein found in the blood, the levels of which rise in response to inflammation (an acute-phase protein). Its physiological role is to bind to phosphocholine expressed on the surface of dead or dying cells (and some types of bacteria) in order to activate the complement system via the C1Q complex.

The term “a disorder associated with oxidative stress” refers to any disorder that results in an increase in the amount of C-reactive protein in blood, such as inflammation or a cardiovascular disorder (e.g., atherosclerosis, coronary heart disease, stroke, and peripheral arterial disease).

The term “tumor” refers to both benign tumor and malignant tumor (e.g., leukemia, colon cancer, prostate cancer, kidney cancer, liver cancer, breast cancer, or lung cancer).

The term “infection” includes viral, bacterial, parasitic, and other microbial infection. Examples of viral infection treatable by this composition include influenza (e.g., Avian influenza or infection with influenza A virus subtype H5N1), severe acute respiratory syndrome (SARS), human immunodeficiency virus (HIV) infection, herpes simplex virus infection, respiratory syncytial virus (RSV) infection, rhinovirus (e.g., human rhinovirus) infection, and coronavirus infection.

The term “glutamate toxicity or excitotoxicity” is the pathological process by which nerve cells are damaged and killed by glutamate and similar substances. This occurs when receptors for the excitatory neurotransmitter glutamate (glutamate receptors) such as the NMDA receptor and AMPA receptor are overactivated. Excitotoxicity may be involved in spinal cord injury, stroke, traumatic brain injury and neurodegenerative diseases of the central nervous system (CNS) such as multiple sclerosis, Alzheimer's disease, amyotrophic lateral sclerosis (ALS), Parkinson's disease, alcoholism or alcohol withdrawal and Huntington's disease. Other common conditions that cause excessive glutamate concentrations around neurons are hypoglycemia and status epilepticus.

The terms “improving,” “enhancing,” “treating,” and “lowering” refer to the administration of an effective amount of a composition of the invention to a subject, who needs to improve one or more of the body conditions or has one or more of the disorders discussed herein, or a symptom or a predisposition of one of more of the disorders or conditions, with the purpose to improve one or more of these conditions, or to prevent, cure, alleviate, relieve, remedy, or ameliorate one or more of these disorders, or the symptoms or the predispositions of one or more of them.

The term “administration” covers oral or parenteral delivery to a subject a composition of the invention in any suitable form, e.g., food product, beverage, tablet, capsule, suspension, and solution.

The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques.

The term “NSAID” refers to non-steroidal anti-inflammatory drug. NSAIDs include the salicylates such as salicylamide and acetylsalicylic acid (aspirin). Non-aspirin NSAIDs include para-aminophenol derivatives such as phenacetin, the pyrazole derivatives such as antipyrine, aminopyrine, dypyrone, nefenamic acid, indomethacin, methimazole, paracetamol, diclophenac sodium/potassium, ibuprofen, naproxen, and ketorolac tromethamine. The analgesic acetaminophen is often categorized as a NSAID even though the compound does not exhibit significant anti-inflammatory activity. Unless otherwise indicated, acetaminophen will be referred to herein as a NSAID.

The term “about” as used herein means ±10% of the indicated numerical value.

This invention is based on the discovery that a pharmaceutical composition comprising a physiologically acceptable salt of dextromethorphan and/or magnesium, quercetin, resveratrol, and hesperidin, as active ingredients exhibits synergistic health benefits, including eliminating the phlegm from the lung, reducing inflammation in the lung, and/or eliminating allergy symptoms in a subject. Contrary to the teaching of administering large quantities of such compounds as resveratrol, a combination of a physiologically acceptable salt of dextromethorphan, magnesium, quercetin, resveratrol, and hesperidin at very low dose results in treating respiratory lung disease and associated phlegm in the lung.

The addition of N-methyl-D-aspartate (NMDA) modulators, physiologically acceptable salt of dextromethorphan and/or magnesium, with quercetin, resveratrol, and hesperidin renders the compositions very effective for reducing oxidative stress in the lung and subsequent elimination of phlegm in the lung. Additional and related unexpected observations include that daily administration of a combination of physiologically acceptable salt of magnesium, with quercetin, resveratrol, and hesperidin maintains the lung almost free of phlegm and allergy symptoms. Further, the addition of magnesium with quercetin, resveratrol, and hesperidin renders the need for these antioxidants at a very low amount without sacrificing the efficacy. This observation is markedly different from the prior art view that these antioxidants have to be administered in large quantities (approximately 1 gram per day) to have any meaningful biological effects. It is to be noted that administration of such large amounts of these anti-oxidants will result in adverse toxicity in the liver resulting in the ineffectiveness of these compounds to treat any meaningful disease.

In some embodiments, the weight ratio between dextromethorphan or physiologically acceptable salts thereof, physiologically acceptable salt of magnesium, and quercetin, resveratrol, and hesperidin or physiologically acceptable salts thereof in a pharmaceutical composition of the invention can be 1.0 parts dextromethorphan: 0.5-10.0 parts magnesium salt: 0.2-3.0 parts quercetin: 0.2-3.0 parts resveratrol: 0.2-3.0 parts hesperidin, or any ratio where the amounts of the ingredients are within their respective ranges. This ratio is abbreviated 1.0:0.5-10.0:0.2-3.0:0.2-3.0:0.2-3.0 (a similar ratio expressing convention is used throughout this specification). For example, in some embodiments, the pharmaceutical composition comprises about 36 mg of dextromethorphan hydrochloride monohydrate (equivalent to about 30 mg of free base), about 100 mg of magnesium sulfate (equivalent to about 40.4 mg of magnesium), about 44.8 mg of quercetin dihydrate (equivalent to about 40 mg of quercetin), about 20 mg of hesperidin and about 40 mg of resveratrol; the corresponding weight ratio of the composition in some embodiments is 1.0:2.78:1.25:0.56:1.12.

Similarly, in some embodiments, the weight ratio between physiologically acceptable salt of magnesium, quercetin, resveratrol, and hesperidin in a composition of the invention may be 1.0:0.1-1.0:0.1-1:0.1-1.0, or any ratio where the amounts of the ingredients are within their respective ranges. In some embodiments, the composition comprises about 100 mg of magnesium sulfate (equivalent to about 40.4 mg of magnesium), about 44.8 mg of quercetin dihydrate (equivalent to 40 mg of quercetin), about 20 mg of hesperidin and about 40 mg of resveratrol and the corresponding weight ratio of the preferred composition is about 1.0:0.45:0.20:0.40.

In some embodiments, a subject can be administered, once or periodically every day, with a maximum total dose of four capsules of the preferred pharmaceutical composition or the composition without dextromethorphan, wherein each capsule contains the above weights of the respective ingredients. After digestion, hesperidin is converted to hesperetin aglycon and other active derivatives, which are absorbed in the body. The quantity of quercetin mentioned above refers to that of quercetin aglycon or the quercetin moiety of a quercetin derivative. Similarly, the quantity of resveratrol refers to resveratrol or the resveratrol moiety in a derivative. The quantity of hesperidin refers to hesperidin itself and not the hesperetin aglycon. The ingredients quercetin, resveratrol and hesperidin can be added to the composition either in a pure form or as an ingredient in a mixture (e.g., a plant extract). Examples of commercially available quercetin and hesperidin include Spectrum Chemicals (Gardena, Calif.).

The composition of this invention can be in various forms such as capsules, tablets, caplets and liquids. For example, the composition containing it can be a soft chew composition that includes physiologically acceptable salt of magnesium, quercetin, to resveratrol, hesperidin, sugar, corn syrup, sucralose, soy lecithin, corn starch, glycerin, palm oil, xylitol, carrageenan, FD&C Yellow #6, FD&C Yellow #5, and natural and/or artificial flavors. An exemplary serving of this soft chew composition (about 5.15 g) includes about 45 mg of quercetin dihydrate, about 100 mg of magnesium sulfate, about 20 mg of hesperidin and about 40 mg of resveratrol. A subject may take one to three servings of this soft chew composition daily. The amounts taken can vary depending on, for example, the disorder or condition to be treated and the physical states of the subject.

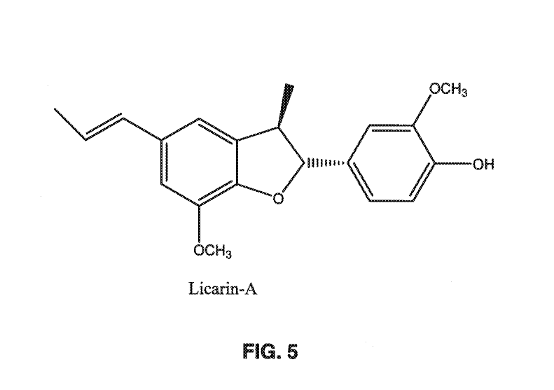

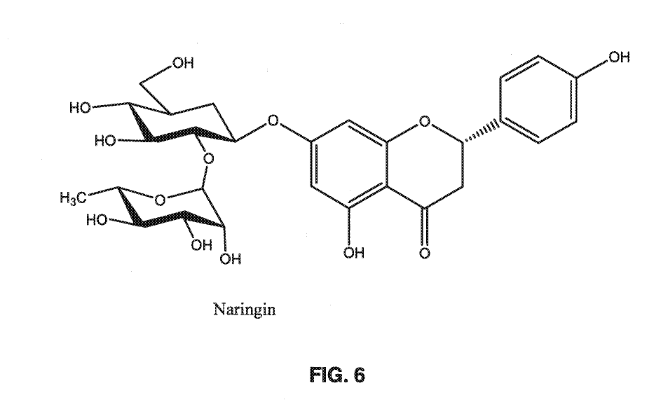

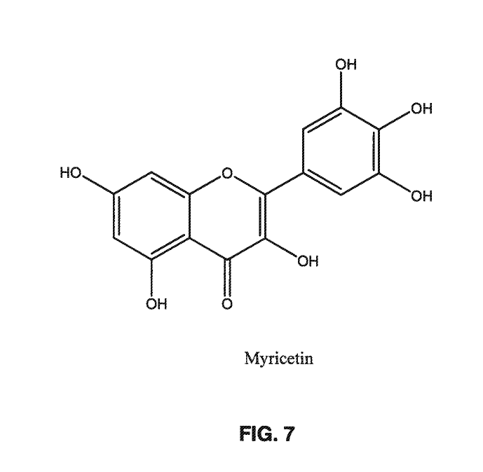

In some embodiments, the composition can further comprise one or more active ingredients, such as an isoflavone (e.g., genistein or genistin), curcumin, isoquercetin, luteolin, epigallocatechin gallate (EGCG), CoQ10, eicosapentaenoic acid (EPA), Licarin-A (Dehydrodiisoeugenol) (FIG. 5), Naringin (FIG. 6), Myricetin (FIG. 7), p-methoxy cinnamic acid and/or docosahexaenoic acid (DHA). These active ingredients can be added to the composition either in a pure form or as a component in a mixture (e.g., an extract from a plant or an animal). A suitable daily dosage of each of these ingredients can vary depending on, for example, the disorder or condition to be treated and the physical states of the subjects. Exemplary daily dosages of some of these ingredients are: about 20-1,000 mg (preferably about 50-200 mg) of curcumin, about 10-100 mg (preferably about 20-50 mg) of Licarin-A, about 10-100 mg (preferably about 10-50 mg) of isoquercetin, about 10-100 mg (preferably about 10-50 mg) of naringin, about 10-100 mg (preferably about 10-50 mg) of myricetin, about 20-200 mg (preferably about 20-50 mg) of EGCG, about 10-300 mg (preferably about 10-25 mg) of genistin/genistein, about 10-300 mg (preferably about 10-50 mg) of luteolin, about 20-200 mg (preferably about 20-50 mg) of EPA, and about 20-300 mg (preferably about 20-50 mg) of DI-IA. Further, it can be sweetened, if necessary, by adding a sweetener such as sorbitol, maltitol, hydrogenated glucose syrup and hydrogenated starch hydrolyzate, high fructose corn syrup, cane sugar, beet sugar, pectin, and/or sucralose. The composition can also contain amino acids, fatty acids, proteins, fibers, minerals, a flavor enhancer, or a coloring agent. Exemplary amino acids include threonine (e.g., L-threonine) and alanine (e.g., L-alanine). Exemplary fatty acids include omega-3 fatty acids (e.g., linolenic acid), omega-6 fatty acids (e.g., linoleic acid), and omega-9 fatty acids (e.g., oleic acid). Exemplary proteins include plant proteins, such as soy proteins and chia seed proteins. Exemplary fibers include plant fibers, such as soy fibers and chia seed fibers. These ingredients can be added in the above-described composition either in a pure form or as a component in a mixture (e.g., an extract from a plant or an animal).

The pharmaceutical composition of the present invention can be combined with NSAIDs to further relieve inflammation. The mechanism of action of NSAIDs is by direct action at the site of tissue injury. NSAIDs peripherally inhibit cyclooxygenases (COX), the enzymes responsible for providing an activated substrate molecule(s) for the synthesis of prostaglandins, which are a group of short-acting mediators of inflammation. The maximal analgesic effect of a standard 325 mg dose of aspirin or of NSAIDs is adjusted to provide the level of pain relief comparable to that achieved by the administration of five milligrams of morphine administered intramuscularly.

When the above-described composition is in powder form, it can be used conveniently to prepare beverages, pastes, jellies, capsules, or tablets. Lactose, microcrystalline cellulose and corn starch are commonly used as diluents for capsules and as carriers for tablets. Lubricating agents, such as magnesium stearate and silicon dioxide are typically included in tablets.

The composition of this invention can be a dietary supplement or a pharmaceutical formulation. As a dietary supplement, additional nutrients, such as minerals or amino acids may be included. The nutraceutical composition can also be a food product. Examples of human food products include, but are not limited to, tea-based beverages, juice, coffee, milk, jelly, cookies, cereals, chocolates, snack bars, herbal extracts, dairy products (e.g., ice cream, and yogurt), soy bean product (e.g., tofu), and rice products.

Without wishing to be bound to any particular theory, the Applicant believes that the compositions of this invention significantly reduce oxidative stress through anti-oxidant as well as modulating signaling pathways and also protects neurons against glutamate toxicity. The compositions may also induce gene expression and activity of T-helper lymphocyte (Th-1) cytokines (e.g., interferon gamma) and may down-regulate T-helper lymphocyte 2 (Th-2) cytokines (e.g., interleukin 13). In addition, they may inhibit the expression and/or activity of one or more of the following three enzymes: matrix metalloproteinase 1 (MMP1), matrix metalloproteinase 2 (MMP2), and cyclooxygenase 2 (COX2). They may also block pathways mediated by epidermal growth factor receptor, such as epiregulin-mediated pathways. The compositions may be used for reducing infection in physically stressed athletes or non-athletes, and improving immune system recovery from intense physical exercises.

The compositions may also be used for treating diseases or disorders, such as a disorder associated with oxidative stress and glutamate toxicity, autoimmune disease (e.g., multiple sclerosis, thyroiditis, rheumatoid arthritis, myositis, lupus, or Celiac disease), skin disease (e.g., eczema, urticaria, or psoriasis), lung disease (asthma, pulmonary fibrosis, or chronic obstructive pulmonary disease), prostatitis, arthritis, tumor, diabetes (type II diabetes), chronic constipation, inflammatory disease (e.g., inflammatory bowel disease such as Crohn's disease or ulcerative colitis), infection, neurodegenerative disease (e.g., dyslexia, dyspraxia, autism, Asperger's disease, Alzheimer's disease, and mild cognitive impairment), and developmental disorder (e.g., attention deficit disorder or attention deficit hyperactivity disorder); for treating brain injury (e.g., physical damages to the brain); for improving concentration or mood; for improving the immune system, and for lowering blood pressure.

Again not wishing to be bound to any particular theory, the Applicant believes that the mechanism for treating viral infection by this composition may include early stage inhibition of viral reproduction by reduction of viral RNA or DNA (e.g., by inhibition of transcription, reverse transcription, and translation). Bacterial infection includes infection by either gram+ or gram− bacteria and infection by either anaerobic or aerobic bacteria. Examples of parasitic infection include leishmaniasis, malaria, and trypanosoma. Other examples of infection include respiratory infection, digestive tract infection, urinary tract infection, blood infection, and nervous system infection.

Further, the compositions may also be used to treat certain symptoms of the above-mentioned diseases or disorders. For example, they may be used to lessen certain symptoms of multiple sclerosis, including muscle weakness, wasting of muscles, pain (such as facial pain or pain without apparent cause), electrical shock sensation, loss of awareness of location of body parts, loss of coordination (such as in speech), shaking when performing fine movements, loss of ability to produce rapidly alternating movement (e.g., movement in a rhythm), and short-term or long term memory loss. As another example, they may be used to reduce the incidence, severity, and/or duration of cold and flu symptoms. In addition, the compositions may also be used as dietary supplements to improve the quality of life of a patient. For example, they may be used to slow the aging process, enhance innate immunity, and improve skin health and digestion.

In addition, the compositions may be used to lessen negative side effects caused by chemotherapy with drugs such as glivec, taxol, and taxotere.

The compositions described above may be preliminarily screened for their efficacy in treating the above-described conditions by in vitro assays and then confirmed by animal experiments and clinical trials. Other suitable analytical and biological assays will be apparent to those of ordinary skill in the art. For example, the bioavailability of quercetin can be measured by conducting pharmacokinetic studies and evaluated by the area under the curve in a plasma-drug concentration time curve.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the appended claims. Several embodiments of the invention are provided in the following Examples. It is to be understood that these are to be considered as exemplary and not limiting.

### EXAMPLES

### Example 1

### Capsule Formulations Containing Dextromethorphan

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | | In 100 | |
|---|---|---|---|---|
| Dextromethorphan Hydrochloride Monohydrate | 36.0 | mg | 3.60 | g |
| Magnesium Sulfate | 100.0 | mg | 10.00 | g |
| Quercetin Dihydrate | 44.8 | mg | 4.48 | g |
| Hesperidin | 20.0 | mg | 2.00 | g |
| Resveratrol | 40.0 | mg | 4.00 | g |
| Microcrystalline Cellulose | 3.1 | mg | 0.31 | g |
| Silicon Dioxide | 4.1 | mg | 0.41 | g |
| Sodium Lauryl Sulfate | 1.0 | mg | 0.10 | g |
| Magnesium Stearate | 1.0 | mg | 0.10 | g |
| Total Solid | 250 | mg | 25.0 | g |

Capsule Formulation 2

| | In each | | In 100 | |
|---|---|---|---|---|
| Dextromethorphan Hydrochloride Monohydrate | 18.0 | mg | 1.80 | g |
| Magnesium Sulfate | 50.0 | mg | 5.00 | g |
| Quercetin Dihydrate | 22.4 | mg | 2.24 | g |
| Hesperidin | 10.0 | mg | 1.00 | g |
| Resveratrol | 20.0 | mg | 2.00 | g |
| Microcrystalline Cellulose | 23.5 | mg | 2.35 | g |
| Silicon Dioxide | 4.1 | mg | 0.41 | g |
| Sodium Lauryl Sulfate | 1.0 | mg | 0.10 | g |
| Magnesium Stearate | 1.0 | mg | 0.10 | g |
| Total Solid | 150 | mg | 15.0 | g |

Capsule Formulation 3

| | In each | | In 100 | |
|---|---|---|---|---|
| Dextromethorphan Hydrochloride Monohydrate | 36.0 | mg | 3.60 | g |
| Quercetin Dihydrate | 44.8 | mg | 4.48 | g |
| Hesperidin | 20.0 | mg | 2.00 | g |
| Resveratrol | 40.0 | mg | 4.00 | g |
| Microcrystalline Cellulose | 13.1 | mg | 1.31 | g |
| Silicon Dioxide | 4.1 | mg | 0.41 | g |
| Sodium Lauryl Sulfate | 1.0 | mg | 0.10 | g |
| Magnesium Stearate | 1.0 | mg | 0.10 | g |
| Total Solid | 160 | mg | 16.0 | g |

### Example 2

Capsule Formulation without Dextromethorphan

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | | In 100 | |
|---|---|---|---|---|
| Magnesium Sulfate | 100.0 | mg | 10.00 | g |
| Quercetin Dihydrate | 44.8 | mg | 4.48 | g |
| Hesperidin | 20.0 | mg | 2.00 | g |
| Resveratrol | 40.0 | mg | 4.00 | g |
| Microcrystalline Cellulose | 29.1 | mg | 2.91 | g |
| Silicon Dioxide | 4.1 | mg | 0.41 | g |
| Sodium Lauryl Sulfate | 1.0 | mg | 0.10 | g |
| Magnesium Stearate | 1.0 | mg | 0.10 | g |
| Total Solid | 240 | mg | 24.0 | g |

### Example 3

### Efficacy of the Combination Therapy in Humans

### Case 1: Condition: Severe Allergy and Constant Phelgm in the Lung

As described in the background, the inventor had suffered from constant allergy reactions from various kinds of allergens as long as he could remember. Due to these allergy reactions, he used to have constant phlegm in the lung and would cough trying to clear the phlegm from the throat and lung. He had undergone all kinds of therapy to get rid of the allergy reactions with very minimal success. A few months ago, he started taking 2 capsules of formulation 1 in Example 1 a day for about 2 weeks and noticed that his allergy symptoms was being reduced every day and the phlegm in the lung was moving upward and cleared every morning. The phlegm tasted differently as compared to the phlegm before the medication. In about 3 weeks, he was completely free of phlegm in the lung and his allergy symptoms were almost gone except some sneezing occasionally. After 3 weeks, he has started taking 2 capsules of formulation 1 in Example 2 every day and since then there is no phlegm in the lung and no allergy reactions. In his view, it is a miracle as he never felt this good and healthy before.

### Case 2: Condition: Aller and Constant Sneezing

A 49 year old white male used to suffer from constant allergic reactions and as a result he used to sneeze constantly as though something is pestering in his nasal passageway. He has started taking 2 capsules of formulation 1 in Example 2 every day and within 10 days, his allergy symptoms had gone completely. He is taking 1 capsule every day as a precaution.

### Case 3: Condition: Fibromyalgia and Allergies

A 42 year old female has body pain and allergy. She used to take narcotics for pain and used to suffer from side effects. In addition, she used to have phlegm in the lung constantly due to allergy. She was given capsules of formulation 1 in Example 2. She wrote the following testimony after taking the capsules. “I was given a capsule called TLI-0326 and another capsule for allergy. I took 2 capsules of TLI-0326 and 2 capsules for allergy every day. Within couple of days, apart from my pain relief, I noticed that phlegm from my lung was moving upwards and I was clearing it every day for a week. The next week I was completely free of phlegm in my lung and I can breathe normally. I am taking the allergy capsule every day to protect my lung. By taking the capsule TLI-0326, my pain problem solved. I sleep through the entire night and can start my mornings with ease. My quality of life, with the other pain medication, was maybe, on a scale of 1 to 10, a 3. The capsule enables me to enjoy life on a 9-10 level. Before taking capsule my morning was a chore to get moving and then spent the rest of the day trying to ease the pain of my muscle discontent.”

### Case 4: Condition: Allergies

A 72 year old dentist used to have constant allergy due to various allergens. He was given the capsule of formulation 1 in Example 2. He made the remark after 4 weeks of taking the 2 capsules per day that his allergy symptoms are almost gone and there is no phlegm in the lung. Currently, he is taking 1 or 2 capsules every day to protect his lung. It is, therefore, apparent that there has been provided, in accordance with the present invention, compositions for treating inflammatory afflictions of the respiratory tract. Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims.

I claim:

1) A pharmaceutical composition comprising effective amounts of dextromethorphan or a physiologically acceptable salt thereof, quercetin, resveratrol, and hesperidin.

2) The pharmaceutical composition of claim 1, wherein the weight ratio between the dextromethorphan or a physiologically acceptable salt thereof, quercetin, resveratrol, and hesperidin is selected from the group consisting of 1:0.5-2:0.3-3.0:0.5-2,0 and 1:0.2-5:0.2-5.0: 0.2-5.0.

3) The pharmaceutical composition of claim 1, wherein the composition is in dry form.

4) The pharmaceutical composition of claim 1, wherein the composition is in liquid form.

5) The pharmaceutical composition of claim 2, wherein the weight ratio between the dextromethorphan or a physiologically acceptable salt thereof, quercetin, resveratrol, and hesperidin is 1:0.2-2:0.3-3.0:0.2-2.0, and wherein the composition further comprises another agent selected from the group consisting of a pharmaceutically acceptable salt of magnesium, genistein, genistin, curcumin, isoquercetin, epigallocatechin gallate (EGCG), eicosapentaenoic acid (EPA), docosahexaenoic acid (DHA), Licarin-A, myricetin, naringin, and a combination thereof.

6) The pharmaceutical composition of claim 5, wherein the other agent is a combination of isoquercetin, EGCG, EPA, myricetin, naringin, and DHA.

7) The pharmaceutical composition of claim 5, wherein the weight ratio between the dextromethorphan or a physiologically acceptable salt thereof, quercetin, resveratrol, hesperidin and the other agent is 1:0.2-2:0.3-3.0:0.5-2.0:0.3-3.0.

8) The pharmaceutical composition of claim 5, wherein the composition is in dry form.

9) The pharmaceutical composition of claim 5, wherein the composition is in liquid form.

10) A nutraceutical or dietary supplement composition comprising a physiologically acceptable salt of magnesium, quercetin, resveratrol, and hesperidin.

11) The nutraceutical or dietary supplement composition of claim 10, wherein the composition is in dry form or liquid form.

12) The nutraceutical or dietary supplement composition of claim 10, wherein the wherein the weight ratio between the physiologically acceptable salt of magnesium, quercetin, resveratrol, and hesperidin is 1:0.2-5:0.2-5.0:0.2-5.0.

13) The nutraceutical or dietary supplement composition of claim 10, wherein the weight ratio between the physiologically acceptable salt of magnesium, quercetin, resveratrol, and hesperidin is 1:0.2-2:0.2-3.0:0.2-3.0, and wherein the composition further comprises another agent selected from the group consisting of genistein, genistin, curcumin, isoquercetin, epigallocatechin gallate (EGCG), eicosapentaenoic acid (EPA), docosahexaenoic acid (DHA), Licarin-A, myricetin, naringin, and a combination thereof.

14) The nutraceutical or dietary supplement composition of claim 13, wherein the other agent is a combination of isoquercetin, EGCG, EPA, myricetin, naringin, and DHA.

15) The nutraceutical or dietary supplement composition of claim 13, wherein the composition is in dry form.

16) The nutraceutical or dietary supplement composition of claim 13, wherein the composition is in liquid form.

17) The composition of claim 13, wherein the composition is a tablet, a capsule, a soft chew, or a gel.

18) A method for treating respiratory disease in a human subject by substantially eliminating phlegm from the lung through administering to the subject in need thereof an effective amount of the composition of claim 1.

19) The method of claim 18, wherein the respiratory disease is selected from the group consisting of lung congestion caused by viral or bacterial infections, allergies, asthma, COPD, eosinophilic cough, bronchitis, sarcoidosis, pulmonary fibrosis, rhinitis and sinusitis.

20) The method of claim 19, wherein the lung congestion caused by viral or bacterial infections is influenza or pneumonia.
